(19) United States
(12) Patent Application Publication
(10) Pub. No.: US 2025/0093273 A1
(43) Pub. Date: Mar. 20, 2025

(54) METHOD AND APPARATUS FOR ESTIMATING THE WATER STRESS OF A PLANT

(71) Applicant: CET ELECTRONICS SNC, Zenson di Piave (IT)

(72) Inventors: Nicola VICINO, Zenson di Piave (IT); Denise VICINO, Zenson di Piave (IT)

(21) Appl. No.: 18/832,609

(22) PCT Filed: Jan. 23, 2023
(86) PCT No.: PCT/IB2023/050549

§ 371 Date: Jul. 24, 2024

(30) Foreign Application Priority Data
Jan. 25, 2022 (IT) 102022000001193

(51) Int. Cl. G01N 21/84; G01N 33/00

(52) U.S. Cl. CPC G01N 21/84; G01N 33/0098; G01N 2021/8466

(57) ABSTRACT

A method is described for estimating the water stress of a plant, wherein the inclination of a leaf, or a set of leaves, of the plant is detected at successive time instants wherein there are different stable illumination conditions on the leaf, and the occurrence of water stress is established or judged when the time course of the observed leaf/leaves inclination(s) corresponds to—or correlates with—a reference time course.

### TECHNICAL FIELD

The invention mainly relates to a method and apparatus for estimating the water stress of a plant.

### BACKGROUND

In agriculture, increased water consumption by evapo-transpiration caused by higher environmental temperatures is observed in plants. On the other hand, lower water availability in soils results in water stress for crops. Then, in order to avoid production declines or plant damage, it is increasingly necessary to irrigate, but the problem is to know when and how much, in order to act promptly and without wasting water.

Known methods involve monitoring soil moisture, climatic parameters, and visual signals from the epigeal part of the plant. With reference to the latter, the onset of water stress is signaled, e.g. in grapevines, by slowed growth of the apices, shorter tendrils, and the angle of leaf inclination. Comparative studies have verified the existence of a correlation between the leaf angle inclination and water stress of the plant, making it highly plausible that leaf angle is an indicator of water stress. However, the above correlation is highly context-dependent and does not seem universally reliable for every plant and condition. Such correlation has been calculated only from diurnal observations and from manual measurements, the making of which is impractical, affected by error due to the personal sensitivity of the operator and based on a normally very small sample of leaves.

### SUMMARY

The main object of the present invention is to provide an improved method and apparatus for determining whether a plant is under water stress. In particular, it is the object of the present invention to provide a method and apparatus, for determining the water stress of a plant, that is automated and/or more reliable.

These and still other objects, which will better appear below, are achieved by an apparatus and method as defined in the appended claims, wherein the dependent claims define advantageous variants thereof.

The Applicant found that the variation in leaf inclination over time is a more reliable indicator of water stress than the absolute value of leaf inclination itself. The Applicant's experimental observations showed that the variation in leaf inclination, that is, the time course of leaf inclination, appears to be associated with different water states of the plant.

Refer to FIG. 1. By convention we define the inclination of a leaf 12 as the angle between a vertical vector V1 that, from any point on the leaf 12, is directed toward the Zenith, and the vector V2 normal to an imaginary plane P that best interpolates the surface of the leaf 12. The vectors V1, V2 have directions exiting from the top blade of the leaf 12 (the one directly exposed to the sun). We will say that a leaf is more inclined than another when it has a greater angle β (in more intuitive terms, a leaf is more inclined if it is more inclined downwards). However, other measurement references are possible, such as the angle complementary to B.

A method for estimating the water stress of a plant is then proposed, wherein
the inclination of a leaf, or preferably a set of leaves, of the plant is detected (i.e. measured) at successive instants of time, and
the occurrence of water stress is established when the time course of the observed leaf/leaves inclination(s) corresponds to—or correlates with—a reference time course. In essence, the time course of the inclination of the leaf/leaves is an indicator of water stress.

In particular, in said successive time instants there is different illumination on the leaf, more in particular stable illumination (i.e. the illumination does not undergo rapid or instantaneous changes but is e.g. the natural and slightly variable illumination caused during the day by sunlight or the almost constant illumination of artificial lighting).

In particular, the occurrence of water stress is determined by comparing the time course of the observed leaf inclination(s) with a reference time course.

Other experimental observations of the Applicant involved the variation of leaf inclination during different illumination conditions, and the resulting time course of leaf inclination appears to be associated with different water states of the plant. Therefore, in a variation of the method
the inclination of a leaf, preferably a set of leaves, of the plant is detected at successive instants of time, and
the occurrence of water stress is established when the leaf is less inclined (β is smaller) in the lower illumination condition than in the higher illumination condition, that is, the occurrence of water stress is established when the leaf is less inclined downward in the lower illumination condition than in the higher illumination condition.

In particular, the Applicant's experimental observations concerned the variation of leaf inclination under daylight illumination and night illumination conditions. Experimental data showed that the greatest range of leaf inclination, and thus greater accuracy or reliability of the method, occurs when leaf inclination detection occurs under stable conditions of full illumination and darkness, e.g. during day and night. Therefore, in a variation of the method
the inclination of a leaf, preferably a set of leaves, of the plant is detected at successive time instants of the day and night when there is different illumination on the leaf, and
the occurrence of water stress is established when the leaf/leaves is/are less inclined in the night illumination condition.

In particular, it is decided that the plant is under water stress condition when the inclination is less at night than during the day.

The decision according to this particular condition is supported by e.g. experimental observations on plants such as wine grapevine and chinese gooseberry. Through day-night comparisons, it has been seen that a plant in optimal water condition tends to minimize leaf inclination during the day and increase it at night, while a plant under water stress tends to increase leaf inclination during the day and decrease it at night. There may be multiple physiological explanations for this phenomenon, depending on the type of plant: attempting to maximize photosynthesis when the water state is optimal, to minimizing water transpiration under water stress conditions, or varying the water potential in leaves as a function of carbohydrate accumulation in tissues.

However, the method can be applied under sunlight or artificial lighting conditions.

To obtain a more reliable estimate, a decision is made for the case of water stress when the leaf inclination with lower illumination is, always with reference to the above definition, less than that with higher illumination in a plurality of consecutive time instants.

Particularly in a favorite variant
the leaf inclination is measured at a first series of consecutive time instants under lower illumination conditions (e.g. at night),
leaf inclination is measured at a second series of consecutive time instants under higher illumination conditions (e.g. during daytime), and
it is decided for the case of water stress when the leaf inclination in the first series of time instants is, always or for the majority of the time instants, less than that in the second set of time instants. That is, e.g. the leaf inclination is measured N times (N≥2) at different instants for lower illumination conditions (e.g. by night), the leaf inclination is measured another N times at different instants for higher illumination conditions (e.g. during the day), and it is decided for the case of water stress when the N measurements of leaf inclination at lower illumination conditions are all less than those measured at higher illumination conditions, or when there are at least K leaf inclination measurements (K<N and e.g. also K>N/2) taken under lower illumination conditions that are smaller than all N measurements taken at higher illumination conditions.

Particularly in another favorite variant
the leaf inclination is measured in a sequence of consecutive time instants at alternating higher or lower illumination conditions for each successive instant (e.g. by night and then during the day), and
it is decided for the case of water stress when leaf inclination is, always or for the majority of the instants, less in the instants with lower lighting conditions.

That is, leaf inclination is measured N times at N different instants under greater or lesser illumination conditions, each time alternated, where N=2*M, M>=1, and
it is decided for the case of water stress when the M measurements of leaf inclination at lower illumination conditions are all less than the M measurements taken at higher illumination conditions, or
when there are at least K leaf inclination measurements (K<M and e.g. also K>M/2) taken under lower illumination conditions that are smaller than all M measurements taken at higher illumination conditions.

In the method, preferably said inclination is detected using an electronic image and/or distance sensor to automate and simplify detection. More preferably, the information regarding leaf inclination is converted into digital data, easily processed by software. Even more preferably, the data emitted by the sensor are processed by a software program, so e.g. one can take advantage of visual recognition algorithms or numerically process the acquired image and/or distance data.

In the method, preferably the occurrence of water stress is established by processing the time course of the observed leaf inclination(s) using an electronic calculation unit or device.

In particular, said processing or calculation takes place
recognizing one or more sample leaves, visible in their entirety or nearly so,
determining the position in three-dimensional space of a plurality of points on each recognized leaf,
calculating an imaginary plane passing through—or interpolating—said plurality of points,
calculating the normal line to the imaginary plane,
considering as inclination of the observed leaf the angle between a vertical vector directed toward the Zenith and the normal to the imaginary plane.

In particular, to improve accuracy, the average of the values acquired for the observed sample leaf inclination is calculated, preferably weighted by the area of the sample leaves. The average thus calculated is taken as the leaf inclination value of the observed plant.

In particular, the position of said plurality of points in three-dimensional space is determined by knowing the distance of each point from the electronic sensor. This is possible if the electronic sensor is, for example, a stereo camera, or a time-of-flight camera (TOF camera), or a structured light system, or a 3D laser scanner associated with a normal camera. Or distance measurement can be avoided and only an electronic image sensor is used (e.g. an RGB camera). A simplified version of the method then involves recognition of the condition of changing leaf inclination from one image to another, as long as the same viewpoint is maintained, by only image analysis algorithms that, from appropriate neural network training, can recognize different leaf perspective features among consecutive images. This version of the method, however, in the absence of pixel distance measurement, does not allow numerical quantification of the inclination angle, thus being less accurate, but could work for recognizing very large excursions of inclination between night and day.

Preferably, in the method when estimating the occurrence of water stress:
a signal is generated, which can be an alarm signal (audible or visual), and/or
irrigation of the plant is activated, in particular an alert for the operator or a signal to automatically activate an irrigation device for the plant, is generated.

In particular, a plant is estimated to be in a water-stressed condition when the inclination, e.g. obtained as the average of several acquisitions, of a group of leaves taken as a reference is less under the condition of lower illumination than their inclination under higher illumination, with the maximum difference evident between day and night.

Preferably the plant is temporarily illuminated at night when capturing the image of one or more leaves, e.g. with a flash system.

Another aspect of the invention concerns an apparatus for estimating the water stress of a plant. The apparatus is equipped with means to perform the above method.

The apparatus comprises:
an electronic image sensor, placed in a fixed position relative to the plant, to detect an image, and preferably also the distance, of a plant leaf,
a logical unit configured for
   reading the data emitted by the electronic sensor,
   processing said data in order to obtain the leaf illumination measurement at successive time instants when there is different stable illumination for the plant, and
   verifying whether the inclination is less in the condition of lower illumination, a case in which it is judged there is water stress.

For example, the electronic sensor is a stereo camera, consisting of two electronic image sensors, spaced a few cm apart (depending on the distance of the object to be detected), to take images of the plant and generate a depth map from which to derive the leaf inclination measurement.

The logical unit preferably is a computer or microprocessor circuit, to which, for example, images are transmitted from the sensor via a data SIM.

Preferably, the device or apparatus comprises means for converting leaf inclination information into digital data, which can be easily managed by software.

To facilitate the comparison of leaf inclination data, the apparatus or sensor comprises a memory to store illumination data acquired at a plurality of different time instants.

Preferably, the apparatus or sensor comprises means for temporarily illuminating the plant, such as a flash system, useful at night.

Preferably, the apparatus or sensor is configured to acquire a sample of plant images at time instants corresponding to stable conditions of higher and lower illumination, e.g. during day and night, framing a representative number of leaves.

In particular, the logic unit is configured to decide that the plant is in a water stress condition when the leaf inclination of a sample of leaves taken as reference is less at the lower illumination condition than at the higher illumination condition, in particular, when said leaf inclination is greater during the day and smaller at night.

For greater accuracy, the logic unit is configured to calculate the average of various leaf inclination data, and the averaged data is considered the leaf inclination value.

To obtain a more reliable estimate, the logic unit is configured to examine image data corresponding to a plurality of consecutive time instants relative to lower or higher illumination. A water stress condition is established when the leaf inclination at all or most of the time instants under lower illumination is less than the leaf inclination at all or most of the time instants under higher illumination.

For the method and apparatus, it is preferable that the time interval between said consecutive instants of detection under different lighting conditions respect the plant's physiological time of adaptation to the new lighting conditions. Then it is convenient that said consecutive instants of detection fall during the middle of the day and during the middle of the night, thus ensuring that the plant has reached a kind of equilibrium position in leaf inclination.

In particular, the logical unit is configured to
automatically recognize one or more sample leaves,
determine the position in three-dimensional space of a plurality of points on each leaf,
compute an imaginary plane passing through or interpolating said plurality of points,
calculate the inclination of the imaginary plane with respect to a reference,
calculate the inclination of the imaginary plane for each observed leaf, and
calculate the average inclination of the imaginary plane for each leaf,
consider the calculated average as a representative data for the plant's leaf inclination.

In particular, the logic unit is configured to determine the spatial position of said plurality of points by determining the distance from the electronic image sensor of the plurality of points.

Preferably the logic unit is configured to generate—and preferably also emit (e.g. via cable or wireless means)—a signal when it decides that a water stress condition is present. Said signal may be an alarm signal (audible or visual), a case in which preferably the logic unit is coupled to, or comprises, a display to show the visual signal, and/or a means for emitting audible sound.

Preferably the apparatus comprises a plant watering device, and the logic unit is configured to issue a warning signal or to automatically activate the watering device when the logic unit estimates there is water stress for the observed plant. More preferably, the logic unit is configured to emit said signal according to phenological data and/or weather forecasts as well, to better estimate the water needs of the plant.

In the method and apparatus:
the electronic sensor is e.g. an analog camera or more preferably a digital camera, associated with a distance measurement system: e.g. the camera is stereoscopic, or of the time-of-flight type (TOF camera), or structured-light type, or associated with a 3D laser scanner; or an RGB camera, and/or
the plant watering device may be, for example, a pump or a sprinkler or a water dispensing device with a remotely controllable solenoid valve; and/or
the leaf inclination is acquired for multiple leaves taken as reference; and/or
the leaf inclination is acquired during the day and at night; and/or
the leaf inclination is acquired for several months; and/or
the leaf inclination is acquired during a production cycle of the plant; and/or
a global datum expressing the overall leaf inclination of the plant is calculated, wherein this datum is the average of the inclinations of said imaginary plane for each observed leaf. The global datum is used to determine whether there is water stress within the decision conditions described above, and/or
methods of detecting the inclination other than described can be used. For example, starting from pixels or detected points of the leaf, one can calculate, instead of the plane P in FIG. 1, the inclination of a segment formed by aligned detected points. Or one can place a goniometer or a graduated angular scale near the leaf and directly read an inclination value.

Another aspect of the invention involves a software for estimating the water stress of a plant. The software comprises program instructions that, when loaded and executed on a computer or microprocessor, perform one or each of the above steps of the method.

### BRIEF DESCRIPTION OF THE DRAWINGS

Further aspects and advantages of the present invention will best appear from the following description of a preferred embodiment illustrated by way of illustration and not limitation with reference to the attached drawings, wherein:

FIG. 1 shows a leaf oriented in space,

FIG. 2 shows a scheme of a detector apparatus,

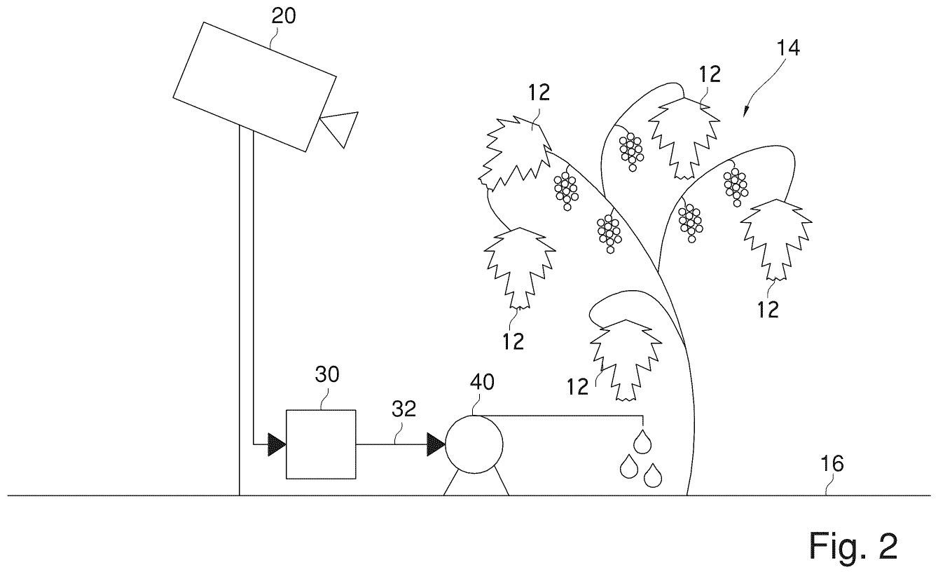

FIG. 3 shows a graph of experimental data for a plant without water stress,

FIG. 4 shows a graph of experimental data for a plant with water stress.

### DETAILED DESCRIPTION

A plant 14, in the illustrated example a vine, is grown on a terrain 16. The plant 14 has leaves 12 that are observed by means of a camera 20 that is in a fixed position in time and that, in a known manner, generates a digital image of the leaves 12. The digital image, formed by pixels, is processed by an electronic circuit 30 to determine the time course of the inclination of the leaves 12.

It is not essential how the digital image is obtained, nor that the image is digital (even a naked-eye observation would suffice at most). But digitization has the advantage of being able to automate the method and take advantage of the computational power of computers and image recognition algorithms.

In particular, it is preferred that the camera 20 be associated with a distance determination sensor/method. For example, the camera 20 is a stereoscopic camera to acquire two images from two slightly different locations on the leaves 12, and exploit the differences in the acquired images to calculate the distance from the camera 20 of the various parts of a leaf 12 and thus construct a depth map. Similar results could be obtained with a TOF camera or structured light systems or, alternatively, with a camera associated with a 3-D laser scanner.

In the acquired image, there is a distinction between the crown of the plant 14, where the leaves 12 are, and the rest of the plant 14.

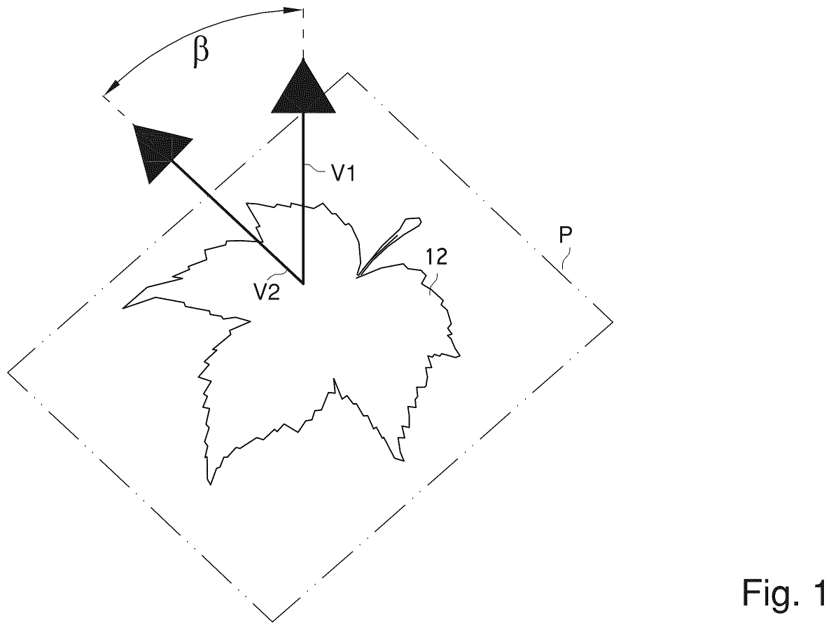

A sample of leaves 12 visible in the image is then identified. For each identified leaf 12, a three-dimensional depth map consisting of points is constructed and the imaginary plane P (see FIG. 1) passing through these points or approximating them (e.g. by linear regression) is calculated. Having determined the imaginary plane P, its geometric inclination is calculated, and e.g. using the convention in FIG. 1, the angle β is taken as the inclination value for the leaf 12.

The observation of leaves 12 occurs, e.g. by day and night, for several months during the growing season.

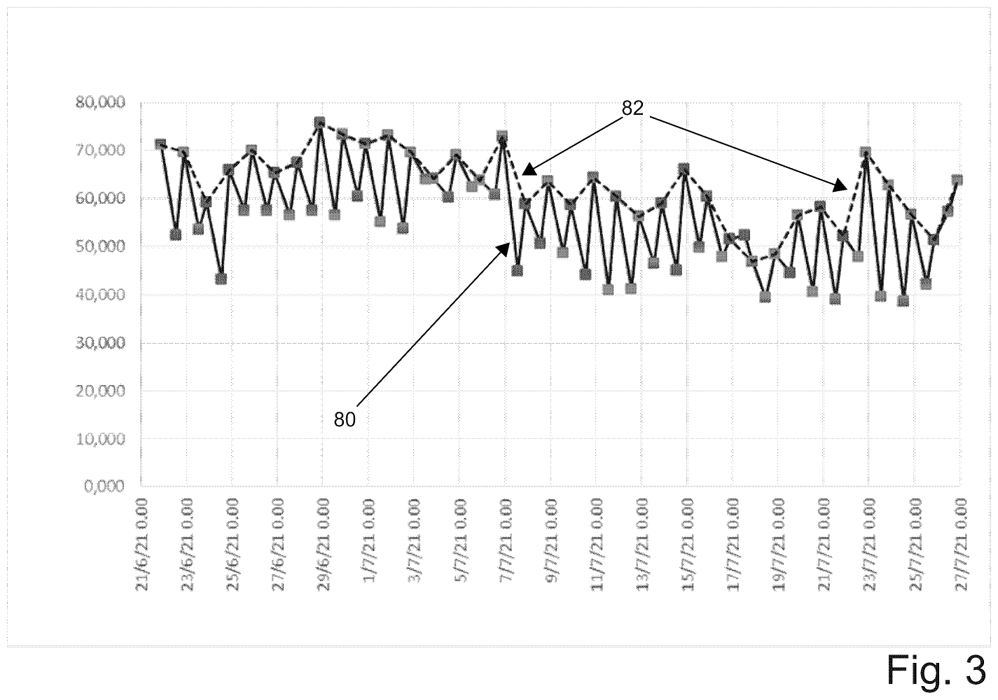

FIG. 3 shows the experimentally detected course 80 for the average inclination of a sample of leaves 12 belonging to an open-field vine plant on which water stress has never been detected. The ordinates of the graph express the angle β, the abscissae the time in days. Represented in this example is leaf inclination measured twice a day, at 3 p.m. (daytime measurement) and 11 p.m. (nighttime measurement, i.e. in the absence of light).

The dotted line 82 joins the points of the course 80 related to nighttime acquisition only (11 p.m.), highlighting that there is almost always a greater inclination at night and less inclination during the day.

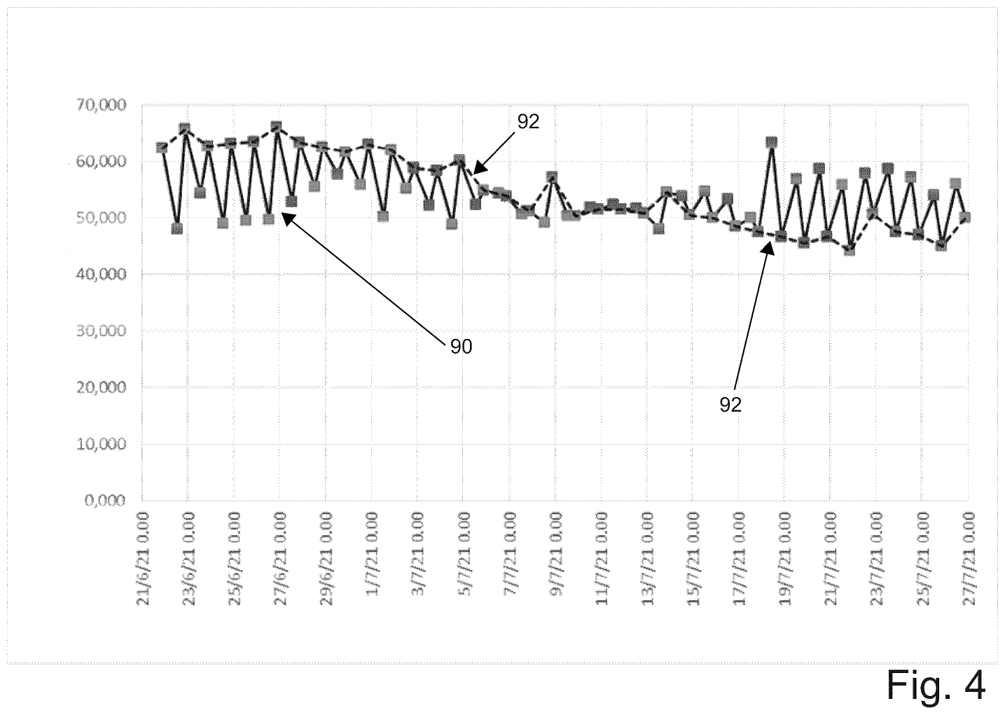

FIG. 4 shows the experimentally detected course 90 for the average inclination of a sample of leaves 12 of another vine plant in the open field, under the same environmental conditions as above, with the exception of water supply (in this case reduced). The graph and acquisitions are organized as in FIG. 3. The plant relative to FIG. 4 from some point later in the season was subject to water stress, as evidenced by simultaneous measurements of water potential made in the field by a Scholander chamber.

The dashed line 92 combines the points of the course 90 related to nighttime acquisition only (11 p.m.). The line 92 highlights that from a certain point in the season onward, after a transitional phase that can be considered a “pre-alarm” condition, a condition of water stress begins in which there is practically always a lower inclination β at night and a higher inclination β during the day. This phenomenon may be associated with the tendency of the plant 14 under stress to limit sun exposure and/or with the decrease in turgor in the supporting tissues of the leaves 12 caused by the shortage of hydration.

Thus, it is believed that compared with the observation of diurnal leaf inclination tout court, whose absolute value is unstable and variable according to weather conditions, the observation of the time course of the leaf inclination β, particularly in day-night variations, is a better parameter for estimating the water stress of the plant 14.

In particular, the detection of the time course of the leaf inclination β comprises the detection during the daytime and also at night.

The electronic circuit 30 can be located in the camera 20, and e.g. transmit image data via cable or wireless means, or be located at a remote location.

In a variant, the electronic circuit 30 is configured to output a signal 32 when it determines the water stress condition. The signal 32 can be exploited to drive a water stress warning device (e.g. a display or sound generator), and/or drive an irrigation device 40, such as solenoid valves, a pump, or a sprinkler. Thus the plant 14 receives water when it needs it and until the water stress condition ceases.

1. Method for estimating the water stress of a plant, wherein
the inclination of a leaf, or a set of leaves, of the plant is detected at successive time instants wherein there are different stable illumination conditions on the leaf, and
the occurrence of water stress is established or judged when the time course of the observed leaf/leaves inclination(s) corresponds to—or correlates with—a reference time course.

2. Method according to claim 1, wherein the inclination of a leaf, or set of leaves, of the plant is detected at successive temporal instants of day and night.

3. Method according to claim 1, wherein the occurrence of water stress is established or judged when the leaf is more downwardly inclined in a higher illumination condition than in a lower illumination condition.

4. Method according to claim 1, wherein
the leaf inclination is measured at a first series of consecutive time instants with a first illumination on the leaf,
the leaf inclination is measured at a second set of consecutive time instants with a second illumination on the leaf, the first illumination being lower than the second illumination, and
a decision is made for the case of water stress when the leaf inclination in the first set of time instants is, always or for the majority of instants, smaller than that in the second set of time instants.

5. Method according to claim 1, wherein the leaf inclination is measured at a sequence of consecutive time instants under alternating, higher or lower, illumination conditions for each successive instant, and
a decision is made for the case of water stress when the leaf inclination is, always or for most of the instants, smaller in the instants with lower illumination conditions.

6. Method according to claim 1, wherein said leaf inclination is detected using an electronic image and/or distance sensor.

7. Method according to claim 6, wherein data emitted from the sensor is processed with a software program to determine or judge the occurrence of water stress.

8. Method according to claim 1, wherein an alert signal is generated when the occurrence of water stress is determined or judged.

9. Method according to claim 1, wherein when the occurrence of the water stress condition is determined or judged, irrigation of the plant is activated.

10. (canceled)

11. Method according to claim 2, wherein the occurrence of water stress is established or judged when the leaf is more downwardly inclined in a higher illumination condition than in a lower illumination condition.

12. Method according to claim 2, wherein
the leaf inclination is measured at a first series of consecutive time instants with a first illumination on the leaf,
the leaf inclination is measured at a second set of consecutive time instants with a second illumination on the leaf, the first illumination being lower than the second illumination, and
a decision is made for the case of water stress when the leaf inclination in the first set of time instants is, always or for the majority of instants, smaller than that in the second set of time instants.

13. Method according to claim 3, wherein
the leaf inclination is measured at a first series of consecutive time instants with a first illumination on the leaf,
the leaf inclination is measured at a second set of consecutive time instants with a second illumination on the leaf, the first illumination being lower than the second illumination, and
a decision is made for the case of water stress when the leaf inclination in the first set of time instants is, always or for the majority of instants, smaller than that in the second set of time instants.

14. Method according to claim 11, wherein
the leaf inclination is measured at a first series of consecutive time instants with a first illumination on the leaf,
the leaf inclination is measured at a second set of consecutive time instants with a second illumination on the leaf, the first illumination being lower than the second illumination, and
a decision is made for the case of water stress when the leaf inclination in the first set of time instants is, always or for the majority of instants, smaller than that in the second set of time instants.

15. Method according to claim 4, wherein
the leaf inclination is measured at a first series of consecutive time instants with a first illumination on the leaf,
the leaf inclination is measured at a second set of consecutive time instants with a second illumination on the leaf, the first illumination being lower than the second illumination, and
a decision is made for the case of water stress when the leaf inclination in the first set of time instants is, always or for the majority of instants, smaller than that in the second set of time instants.

16. Method according to claim 2, wherein the leaf inclination is measured at a sequence of consecutive time instants under alternating, higher or lower, illumination conditions for each successive instant, and
a decision is made for the case of water stress when the leaf inclination is, always or for most of the instants, smaller in the instants with lower illumination conditions.

17. Method according to claim 3, wherein the leaf inclination is measured at a sequence of consecutive time instants under alternating, higher or lower, illumination conditions for each successive instant, and
a decision is made for the case of water stress when the leaf inclination is, always or for most of the instants, smaller in the instants with lower illumination conditions.

18. Method according to claim 11, wherein the leaf inclination is measured at a sequence of consecutive time instants under alternating, higher or lower, illumination conditions for each successive instant, and
a decision is made for the case of water stress when the leaf inclination is, always or for most of the instants, smaller in the instants with lower illumination conditions.

19. Method according to claim 4, wherein the leaf inclination is measured at a sequence of consecutive time instants under alternating, higher or lower, illumination conditions for each successive instant, and
a decision is made for the case of water stress when the leaf inclination is, always or for most of the instants, smaller in the instants with lower illumination conditions.

20. Method according to claim 5, wherein the leaf inclination is measured at a sequence of consecutive time instants under alternating, higher or lower, illumination conditions for each successive instant, and
a decision is made for the case of water stress when the leaf inclination is, always or for most of the instants, smaller in the instants with lower illumination conditions.
